(19) United States
(12) Patent Application Publication
(10) Pub. No.: US 2010/0049035 A1
(43) Pub. Date: Feb. 25, 2010

(54) Brain image segmentation from ct data

(76) Inventors: Qingmao Hu, Singapore (SG); Wieslaw Lucjan Nowinski, Singapore (SG); Guoyu Qian, Singapore (SG); Aamer Aziz, Singapore (SG)

Correspondence Address: STRAUB & POKOTYLO, 788 Shrewsbury Avenue, TINTON FALLS, NJ, 07724, US

(21) Appl. No.: 11/921,122

(22) PCT Filed: Aug. 25, 2005
(86) PCT No.: PCT/SG2005/000290

§ 371 Date: Sep. 25, 2009

Related U.S. Application Data
(60) Provisional application No. 60/685,175, filed on May 27, 2005.

(51) Int. Cl. A61B 5/05; G06K 9/00

(52) U.S. Cl. 600/425; 382/131

(57) ABSTRACT

The brain structure is extracted from CT data based on thresholding and brain mask propagation. Two thresholds are determined: a high threshold excludes the high intensity bones, while a low threshold excludes air and CSF. Brain mask propagation uses the spatial relevance of brain tissues in neighbouring slices to exclude non-brain tissues with similar intensities.

### FIELD OF THE INVENTION

This invention relates to image segmentation of the brain using computed tomography (CT) scan data.

### BACKGROUND

Some of the biggest advancements in medical sciences have been in diagnostic imaging. With the advent of multi-detector CT scanners and faster scan times, CT has become the centerpiece for cranial imaging. It is the examination modality of choice for investigating stroke, intracranial haemorrhage, trauma and degenerative diseases. It is readily available, has few contraindications, and offers rapid results and acceptably high sensitivity and specificity in detecting intracranial pathologies.

CT has several advantages over magnetic resonance imaging (MRI). These include short imaging times (about 1 second per slice), widespread availability, ease of access, optimal detection of calcification and haemorrhage (especially subarachnoid haemorrhage), and excellent resolution of bony detail. CT is also valuable in patients who cannot have MRI because of implanted biomedical devices or ferromagnetic foreign material.

The brain consists of gray matter (GM) and white matter (WM including in cerebrum, cerebellum and brain stem. In CT brain images, bones have the highest intensity, followed by GM, WM, cerebrospinal fluid (CSF), and air. Non-brain tissues like various sinuses and muscles may have similar intensities to GM or WM. Due to the invasive nature of CT imaging, the slice thickness is normally large (>=5 mm) to decrease the subject's exposure to radiation. The implication of the large slice thickness is that neighbouring axial slices have some relationship, but it cannot be assumed that the brain tissues as a whole will form the largest connected component as in the case of MRI with small slice thickness.

Literature on brain segmentation from CT images is very sparse.

Maksimovic et al 2000 used active contours models to find lesions and ventricles in patients with acute head trauma with manual drawing of initial contours. [Maksimovic R, Stankovic S, Milovanovic D. Computed tomography image analyzer: 3D reconstruction and segmentation applying active contour models—‘snakes’. International Journal of Medical Informatics 2000; 58-59: 29-37.]

Deleo et al 1985 proposed a semi-automatic method to do brain segmentation from CT images. Users were requested to manually select representative points of cerebrospinal fluid (CSF), gray matter (GM), and white matter (WM) in the region superior to the third ventricle (7 consecutive axial slices, lowest one containing the third ventricle) to avoid beam hardening. Thresholds are calculated based on the manual specification of the representative CSF, GM and WM to distinguish between CSF and WM, and WM and GM. This solution has serious problems for it to be considered feasible: manual specification is tedious and error prone without training, the beam hardening cannot be handled, not all the brain is covered for categorization, and spatial information is not exploited to deal with tissues having overlapped intensity. [Deleo J M, Schwartz M, Creasey H, Cutler N, Rapoport S I. Computer-assisted categorization of brain computerized tomography pixels into cerebrospinal fluid, white matter, and gray matter. Computers and Biomedical Research 1985; 18: 79-88.]

Ruttimann et al 1993 proposed to use maximum between class variance criteria for differentiating hard and soft tissues, and CSF was segmented using a local thresholding technique based on maximum-entropy principle. The processing is limited to selected axial slices and no spatial relationship between neighbouring slices is considered. (Ruttimann U E, Joyce E M, Rio D E, Eckardt M J. Fully automated segmentation of cerebrospinal fluid in computed tomography. Psychiatry Research: Neuroimaging 1993; 50: 101-119]

Soltanian-Zadeh and Windham 1997 proposed to find brain contours in a semi-automatic way: manually specify the thresholds at different regions to binarize CT slices, use edge tracking to find contours, use multi-resolution to resolve broken contours, and specify seed points to pick up the desired contour. This is basically a manual method, and the vast amount of user intervention is its major drawback. [Soltanian-Zadeh H. Windham J P. A multiresolution approach for contour extraction from brain images. Medical Physics 1997; 24(12): 1844-1853.]

There are, however, certain limitations to CT scanning of the head. The artifacts that arise due to beam hardening and spiral off-center can be serious enough to produce misdiagnosis. There is a radiation burden on the patient and pregnancy is a contraindication. The tissue contrast is not high enough to identify or segment various cerebral tissues adequately. This is a major drawback when advanced image processing and segmentation is required.

The present invention is directed to overcoming or at least reducing the drawbacks of CT scanning mentioned.

### SUMMARY

In broad terms, the brain structure is extracted from CT data based on thresholding and brain mask propagation. Two threshold values are determined: a high threshold excludes the high intensity bones, while a low threshold excludes air and CSF. Brain mask propagation is the use of the spatial relevance of brain tissues in neighbouring slices to exclude non-brain tissues with similar intensities.

The invention provided a method for generating a segmented brain image from a 2-dimensional slice computed tomography (CT) scan data set, comprising the steps of:
(a) choosing a reference slice of said CT data, and for said reference slice:
   determining a region of interest;
   determining a low threshold value from intensity values of said reference slice within said region of interest; and
   determining a high threshold value from intensity values of said reference slice within said region of interest; and
(b) for each slice in said data set:
   determining a region of interest;
   performing a binarization of said slice components by use of said low threshold value and said high threshold value to give foreground connected components; and
   excluding those foreground connected components that do not satisfy a spatial relevance criterion with reference to an adjacent slice.

The invention further provides apparatus for generating a segmented brain image, comprising:
(a) a computed tomography (CT) scanner producing a CT scan data set;
(b) a processor.
   generating 2-dimensional slice data from said data set;
   for a reference slice of said CT data: determining a region of interest, determining a low threshold value from intensity values of said reference slice within said region of interest, and determining a high threshold value from intensity values of said reference slice within said region of interest; and
   for each slice in said data set: determining a region of interest, performing a binarization of said slice components by use of said low threshold value and said high threshold value to give foreground connected components; and excluding those foreground connected components that do not satisfy a spatial relevance criterion with reference to an adjacent slice; and
(c) a display device to display the non-excluded foreground connected components in each said slice as said segmented brain image.

### DESCRIPTION OF THE DRAWINGS

FIG. 1 shows the flow chart of the disclosed method.

FIG. 2 shows the reference image which is an axial slice around the anterior and posterior commissure with the third ventricle present and without the orbits.

FIG. 3 shows a flow chart for finding a region of interest.

FIGS. 4A and 4B show the space enclosed by the skull of the reference image, and the region of interest of the reference image, respectively.

FIG. 6 shows a flow chart for finding a low threshold.

FIG. 6 shows a flow chart for finding a high threshold.

FIGS. 7A and 7B show thresholding of the reference image and the region of interest within the reference image with low and high thresholds to get the binary mask.

FIGS. 8A and 8B show brain candidates for the reference image and its region of interest from the binary mask using distance criteria to exclude skull.

FIGS. 9A and 9B show brain candidates for another axial slice and its region of interest, determined using distance criteria.

FIG. 10 is A flow chart of brain mask propagation.

FIGS. 11A and 11B show the derived brain after propagation of brain masks.

FIG. 12 shows a schematic block diagram of a computer hardware architecture on which the methods can be implemented.

### DETAILED DESCRIPTION

The coordinate system (xyz) used herein follows the standard radiological convention: x runs from the subject's right to left, y from anterior to posterior, and z from superior to inferior. The intensity of a voxel (x, y, z) is denoted as g(x, y, z). An axial slice consists of those voxels with z being a constant.

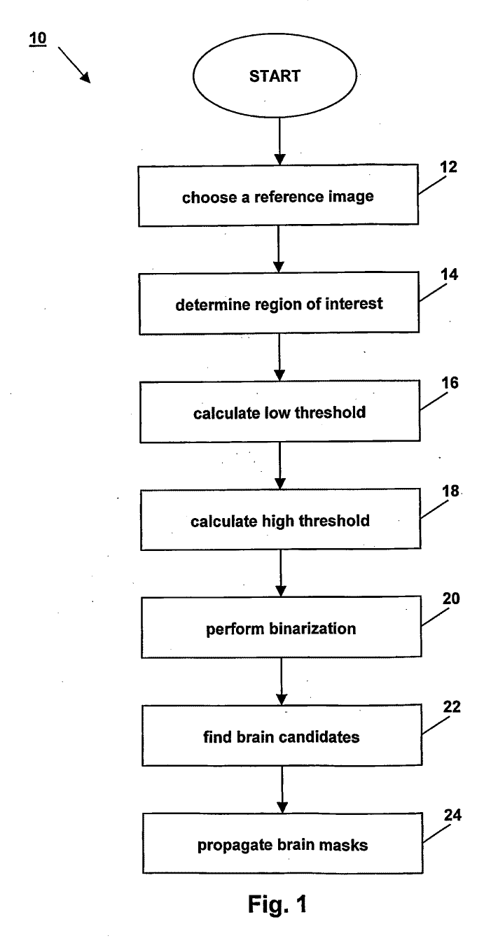

FIG. 1 shows the flow chart of the disclosed method 10 of producing brain segmentation images, and assumes a 3D volumetric CT data set obtained from a scanner in the usual manner.

Choose a Reference Image g(x, y, z₀) (Step 12)

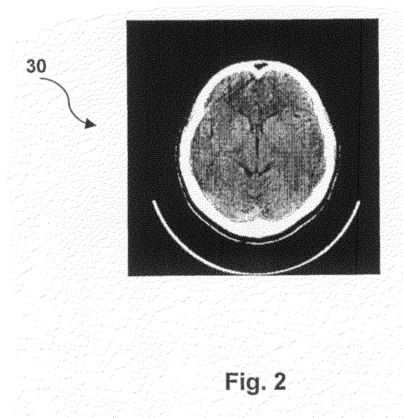

The reference image is a 2D image obtained from the 3D volumetric CT data set to be binarized. The reference image should have the following characteristics: it has WM, GM, CSF, air, and skull tissues present; it is easily extracted from the volume anatomically; the proportion of GM and WM should be stable. One suitable reference image is the axial slice passing through the anterior and posterior commissures. In practice, this reference image can be approximated by an axial slice 30 with third ventricle present and without eyes, as shown in FIG. 2. The axial slice number is denoted as z₀, and the reference slice is denoted as g(x, y, z₀).

Determine Region of Interest (Step 14)

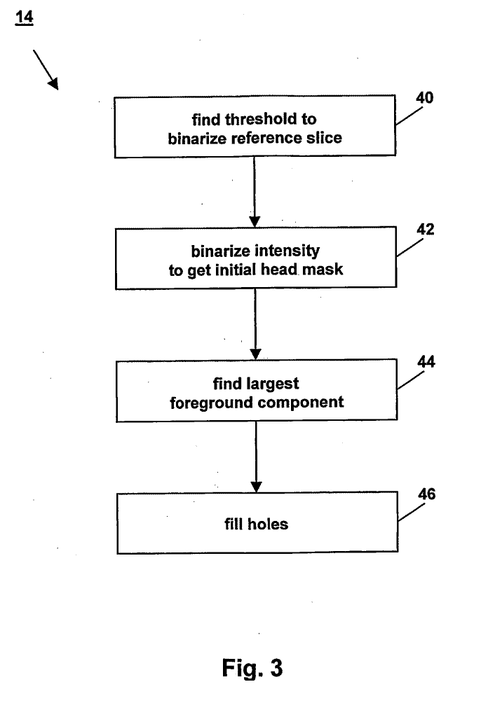

As it is the brain tissues within the skull that is of interest, the region of interest (ROI) of the reference image 30 is the space enclosed by the skull, and is called the ‘head mask’ hereinafter. The region of interest (ROI) can be achieved through the following sub-steps as shown in FIG. 3:
1) Find the threshold to binarize the reference slice (step 40). From the intensity histogram of the volume g(x, y, z), classify the intensity into 4 clusters (corresponding to air, CSF, WM/GM, and bone) using known fuzzy C-means (FCM) clustering, with the first cluster having the smallest intensity. The maximum intensity of the first cluster plus a constant of around 5 is denoted as ‘backG’.
2) Binarize g(x, y, z₀) to get initial head mask ‘skullM(x, y, z₀)’: if g(x, y, z₀) is smaller than backG, then skullM(x, y, z₀) is set to 0 (background), otherwise skullM(x, y, z₀) is set to 1 (foreground) (step 42).
3) Find the largest foreground connected component of skullM(x, y, z₀) (step 44), being the foreground connected component having the largest number of foreground pixels.
4) Fill the holes within skullM(x, y, z₀) (step 46). Any background component completely enclosed by foreground components is considered a hole and is set to foreground.

In this way, all pixels enclosed by the skull are located.

FCM clustering (i.e. step 40) is used in preference to curve fitting of the intensity histogram, as the former does not assume a Gaussian distribution and will be valid even in the presence of heavy noise and other artifacts.

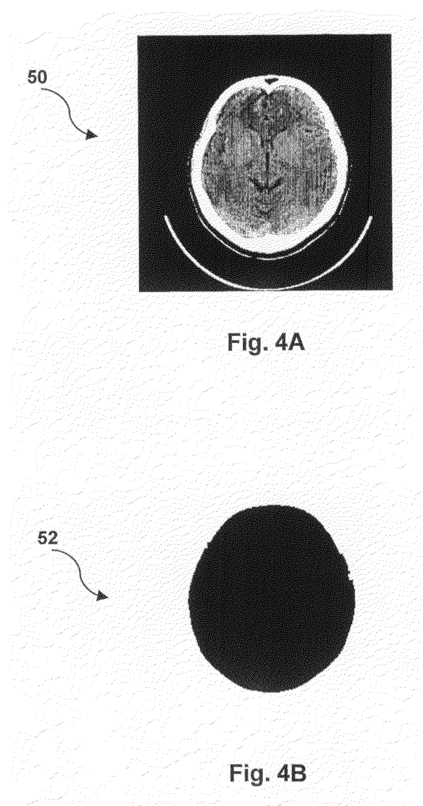

FIG. 4A shows the reference image 50, and FIG. 4B shows the corresponding determined region of interest (ROI 52).

Calculate Low Threshold (Step 16)

The low threshold value is used to exclude air and CSF from the brain image, and is determined by the following sub-steps, shown in FIG. 5:
1) From the intensity histogram of the reference image g(x, y, z₀) in the skull mask skullM(x, y, z₀), classify the intensity into 4 clusters corresponding to air and CSF, WM, GM, and bone (step 60). Cluster 1 represents the air and CSF components. (The smallest intensity of the fourth cluster is denoted as ‘minione’, which will be used for determination of the high threshold value.)
2) The low threshold value (lowThresh) is now calculated (step 62) as: lowThresh=meanC₁+α₁*sdC₁, where meanC₁ and sdC₁ are the mean and standard deviation of cluster 1, while α₁ is a constant in the range of 0 and 3. When it is required to have less brain tissues classified as non-brain tissues, α₁ should be small, say, less than 1; otherwise, if more interested in separating brain from non-brain tissues, α₁ should be big, say greater than 2.

Calculate High Threshold (Step 18)

As mentioned, the high threshold value serves to exclude bone (that is brighter than both GM and WM. Due to large slice thickness and partial volume effect, it can appear that the bright bone is spatially adjacent to GM and WM, though physically bones are not exactly adjacent to GM or WM. This spatial relationship is utilized to determine the high threshold.

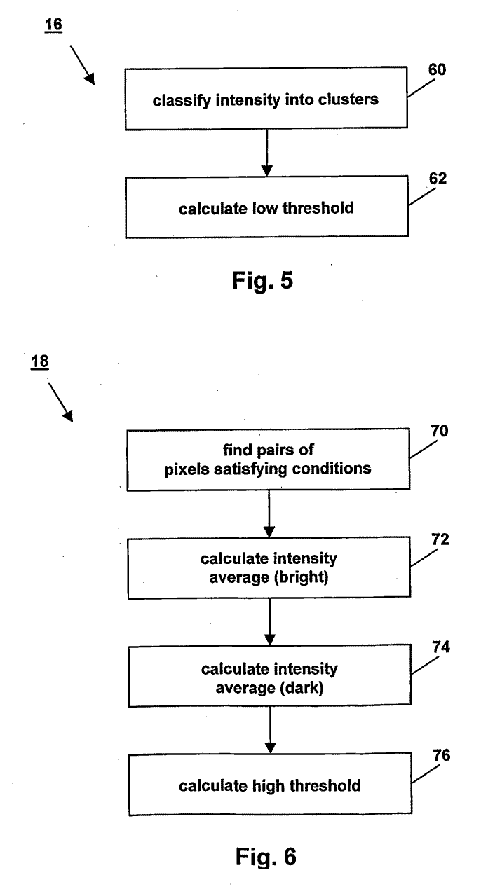

The high threshold value is determined from pairs of pixels in the reference image 50. Each pair of pixels is 8-connected. One pixel is bone while the other pixel is either WM or GM. The high threshold value is obtained through the following sub-steps, shown in FIG. 6:
1) Within the head mask skullM(x, y, z₀) of the reference image find all pairs of pixels satisfying: a) the pair is 8-connected; b) the intensity of one pixel is not smaller than minBone (corresponding to a bone pixel) and c) the intensity of the other is smaller than minBone but greater than lowThresh (corresponding to a GM or WM pixel) (step 70).
2) For all the pairs of pixels found in 1), calculate the intensity average of pixels with intensities not smaller than minBone and denote it as brightAvg (step 72). Similarly, calculate the intensity average of pixels with intensities smaller than minbone, and denote it as darkAvg (step 74).
3) The high threshold is determined by highThresh=α brightAvg+(1−α)darkAvg. Here a is a constant in the range of 0 and 1. If the cost to exclude brain tissue is greater than the cost to include non-brain tissue in the segmentation, a should be greater than 0.5. If both costs are equally important or a minimum classification error is required, then a should be 0.5 (step 76).

Perform Binarization (Step 20)

Binarization is performed on the original CT volume g(x, y, z) to get the binary mask binM(x, y, z) by the following formula

$$binM ( x , y , z ) = \{ \begin{matrix}1 , & lowThresh \leq g ( x , y , z ) \leq highThresh \\ 0 , & otherwise\end{matrix}$$

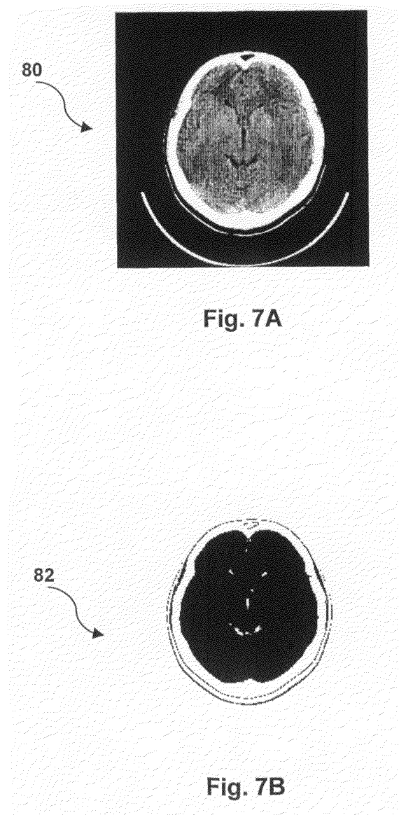

Binarization yields the foreground and background pixels. FIG. 7A shows the reference image 80, as FIG. 7B shows its resultant binary mask 82.

Find Brain Candidates (Step 22)

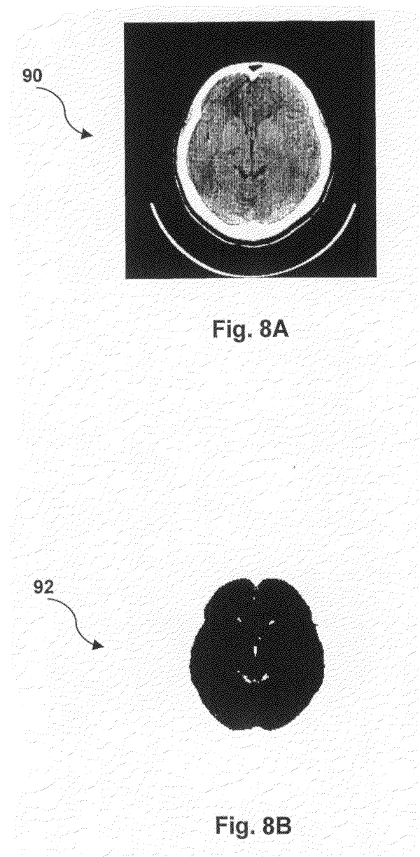

For all axial slices, their head masks are found as described in step 14. By the process of step 20, for any axial slice z, the foreground connected components of binM(x, y, z) (i.e. having value=1) are found. The boundary pixels of the head mask are those foreground pixels within the 3×3 neighbourhood where there is at least one background pixel. When the distance to the boundary of the head mask of this axial slice is large enough, then the component is not skull. When the smallest distance to the head mask of the axial slice is larger than a constant (say, 10 mm), the component is taken as the brain candidate; if otherwise, then the foreground component is set to background. FIG. 8A shows the brain candidates of the reference image 90, and FIG. 8B shows the brain candidates of the ROI 92.

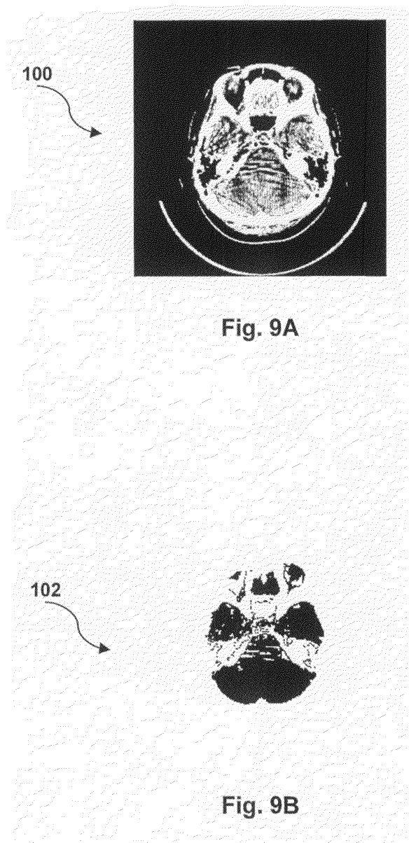

FIG. 9A shows the brain candidates of an axial slice inferior to the reference image 100, and FIG. 9B shows the brain candidates of the ROI 102. Note that in FIGS. 9A and 9B, for axial slices inferior to the reference image, there are still non-brain tissues (like extraocular muscles) remaining as brain candidates.

Propagate Brain Masks (Step 24)

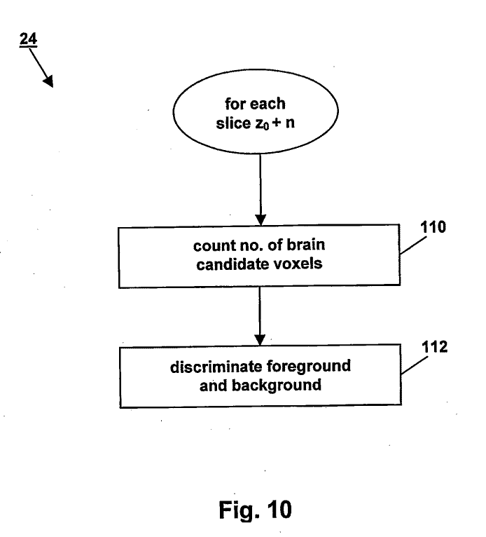

The non-brain regions can be removed through brain mask propagation. Specifically, all the brain candidates with z>z₀ are checked consecutively starting from slice z₀+1. As shown in FIG. 10, in slice z₀+1, all the foreground components are checked in the following way.
1) For a foreground connected component at slice z₀+1, suppose the number of brain candidate pixels is N, and the connected component is a point set:

$${⋃}_{i} ( {x}_{i} , {y}_{i} , {z}_{0} + 1 ) .$$

For all (xᵢ, yᵢ), count the number of brain candidate voxels (xᵢ, yᵢ, z₀) at slice z₀ and denote it as N₁ (step 110).
2) If Nᵢ is smaller than a proportion of N, then the connected component at slice z₀+1 is very different from the brain contents at the superior axial slice z₀, and this foreground connected component at slice z₀+1 is turned to background (step 112). Specifically, when N₁ is smaller than β*N, the foreground connected component at slice z₀+1 is turned to background. Here β is a constant in the range of 0 to 1, typically it takes the value of 0.5.

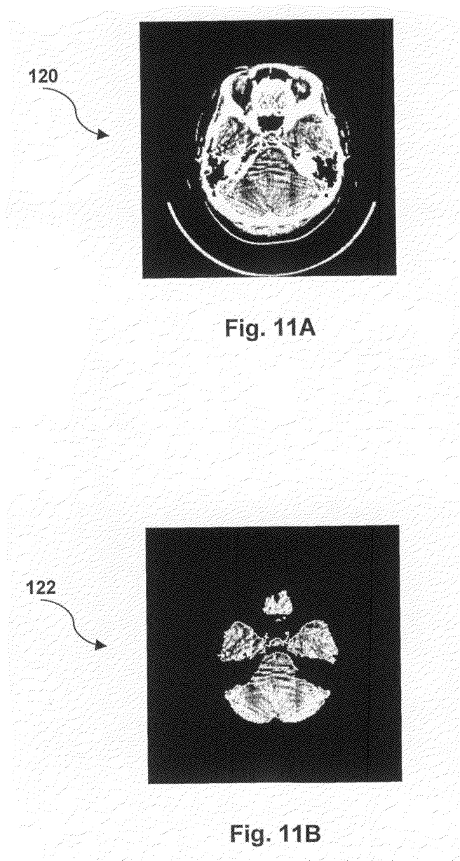

After all the foreground connected components in slice z₀+1 are checked, the process proceeds to slice z₀+2. The procedure as performed in slice z₀+1 is repeated, taking slice z₀+1 as the comparing reference to count N₁. This process continues until all the axial slices with z greater than z₀ have been checked. The resultant remaining brain candidates are the brain tissue. FIGS. 11A and 11B show the eventual brain images 120, 122 after the brain propagation of the axial slice shown in FIGS. 9A and 9B.

Computer Hardware

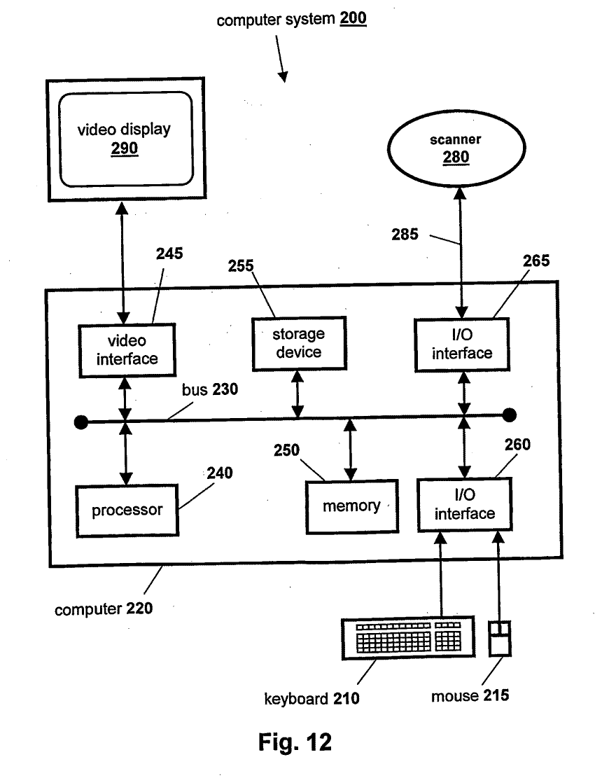

FIG. 12 is a schematic representation of a computer system 200 suitable for executing computer software programs that perform the methods described herein. Computer software programs execute under a suitable operating system installed on the computer system 200, and may be thought of as a collection of software instructions for implementing particular steps.

The components of the computer system 200 include a computer 220, a keyboard 210 and mouse 215, and a video display 290. The computer 200 includes a processor 240, a memory 250, input/output (I/O) interface 260, communications interface 265, a video interface 245, and a storage device 255. All of these components are operatively coupled by a system bus 230 to allow particular components of the computer 220 to communicate with each other via the system bus 230.

The processor 240 is a central processing unit (CPU) that executes the operating system and the computer software program executing under the operating system. The memory 250 includes random access memory (RAN, and read-only memory (ROM), and is used under direction of the processor 240.

The video interface 245 is connected to video display 290 and provides video signals for display on the video display 290. The displayed images include the various axial slice pixels/voxels described above. User input to operate the computer 220 is provided from the keyboard 210 and mouse 215. The storage device 255 can include a disk drive or any other suitable storage medium.

The computer system 200 receives data from a CT scanner 280 via a communications interface 265 using a communication channel 285.

The computer software program may be recorded on a storage medium, such as the storage device 255. A user can interact with the computer system 200 using the keyboard 210 and mouse 215 to operate the computer software program executing on the computer 220. During operation, the software instructions of the computer software program are loaded to the memory 250 for execution by the processor 240.

Other configurations or types of computer systems can be equally well, used to execute computer software that assists in implementing the techniques described herein.

Conclusion

Embodiments of the invention are advantageous in that they are automatic and can handle various artifacts well to provide a robust segmentation.

1. A method for generating a segmented brain image from a 2-dimensional slice computed tomography (CT) scan data set, comprising the steps of: (a) choosing a reference slice of said CT data, and for said reference slice: determining a region of interest; determining a low threshold value from intensity values of said reference slice within said region of interest; and determining a high threshold value from intensity values of said reference slice within said region of interest; and
(b) for each slice in said data set: determining a region of interest; performing a binarization of said slice components by use of said low threshold value and said high threshold value to give foreground connected components; and excluding those foreground connected components that do not satisfy a spatial relevance criterion with reference to an adjacent slice.

2. A method according to claim 1, wherein said foreground connected components are those components having an intensity value falling between said low threshold value and said high threshold value.

3. A method according to claim 2, wherein said spatial relevance criterion is based on the number of foreground connected pixels in said slice being greater than a proportion of foreground connected pixels in said adjacent slice.

4. A method according to claim 3, wherein said excluding step includes determining brain candidate components from said foreground connected components by excluding those foreground connected components that are less than a predetermined distance from the skull defined as a brain mask boundary before applying said spatial relevance criterion.

5. A method according to claim 4, wherein said head mask boundary is determined with reference to . . . (p. 8)

6. Apparatus for generating a segmented brain image, comprising: (a) a computed tomography (CT) scanner producing a CT scan data set;
(b) a processor: generating 2-dimensional slice data from said data set; for a reference slice of said CT data: determining a region of interest, determining a low threshold value from intensity values of said reference slice within said region of interest, and determining a high threshold value from intensity values of said reference slice within said region of interest; and for each slice in said data set: determining a region of interest, performing a binarization of said slice components by use of said low threshold value and said high threshold value to give foreground connected components; and excluding those foreground connected components that do not satisfy a spatial relevance criterion with reference to an adjacent slice; and
(c) a display device to display the non-excluded foreground connected components in each said slice as said segmented brain image.

7. Apparatus according to claim 6, wherein said processor determines said foreground connected components to be those components having an intensity value falling between said low threshold value and said high threshold value.

8. Apparatus according to claim 7, wherein said processor determines said spatial relevance criterion based on the number of foreground connected pixels in said slice being greater than a proportion of foreground connected pixels in said adjacent slice.

9. Apparatus according to claim 8, wherein said processor excludes brain candidate components from said foreground connected components by excluding those foreground connected components that are less than a predetermined distance from the skull defined as a brain mask boundary before applying said spatial relevance criterion.

10. Apparatus according to claim 9, wherein said head mask boundary is determined with reference to those foreground pixels within the neighbourhood of pixels where there is at least one background pixel.

11. Image data carried on a storage medium produced according to the method of claim 1.

12. Image data carried on a storage medium produced according to the method of claim 2.

13. Image data carried on a storage medium produced according to the method of claim 3.

14. Image data carried on a storage medium produced according to the method of claim 4.

15. Image data carried on a storage medium produced according to the method of claim 5.
